(19) United States
(12) Patent Application Publication
(10) Pub. No.: US 2013/0244191 A1
(43) Pub. Date: Sep. 19, 2013

(54) Lighter Eyez

(71) Applicants: Jeffrey George Bush, Port St. Lucie, FL (US); Joshua Tyler Kay, Port St. Lucie, FL (US)

(72) Inventors: Jeffrey George Bush, Port St. Lucie, FL (US); Joshua Tyler Kay, Port St. Lucie, FL (US)

(21) Appl. No.: 13/356,909

(22) Filed: Mar. 16, 2012

(51) Int. Cl. F23Q 2/32; A61P 27/02; A61K 35/00

(52) U.S. Cl. 431/253; 514/789

(57) ABSTRACT

A combination cigarette lighter and eye-drop applicator wherein the eye-drop device is secured to the lighter by being press fit secured with plastic clips or secured by hollow housing, a reservoir in the housing adapted to hold a supply of eye-drops and a squeeze bottle for applying the eye-drops in addition to the fire-lighting section of ordinary lighters, such that a person could ease eye irritation and redness due to smoke or allergies without needing anything but a lighter.

### FIELD OF INVENTION

The present invention relates, in general, to cigarette lighters and eye-drop applicators and, more particularly, this invention relates to a combination cigarette lighter and redness relief eye-drop applicator.

### BACKGROUND OF INVENTION

As is generally well known, smokers use eye-drops to alleviate redness due to minor eye irritations. Eye-drops are generally available as a squeeze bottle or pop-tab drop applicator. Either type must be carried in a handbag or pocket separately from a gas lighter which is predominantly used by smokers and drinkers alike. Efforts have not been made until now to overcome disadvantages of carrying redness reliever eye-drops separately from the gas lighter. The lighter device provides a housing for receiving a portion of the eye-drops secured to the lighter by plastic press fit clips. Additionally, a cover structure is provided to prevent discharge of the eye-drop device when not in use. The eye-drop applicator, which contains plenty of redness relief solution, is adapted to be removable from the lighter device for applying eye-drops as necessary.

### SUMMARY OF INVENTION

The present invention provides a combination lighter device and eye-drop applicator for disposable use, although the same technology could be incorporated into a ZIPPO (registered trademark) style lighter for refillable use. This combination includes a slide, press fit or non-detachable hollow housing, which would hold a predetermined quantity of eye-drops for easy accessibility and a second hollow housing which would hold a predetermined quantity of lighter fluid. An identification plate attached to the upward forward portion of the device for easy personalization. The instant invention resolves the problems in an efficient and cost effective manner. Additionally gives the user access to both the lighter and the eye-drop device with no interference and the ease of convenience.

### BRIEF DESCRIPTION OF DRAWINGS

FIG. 1 is a perspective view of the combined cigarette lighter and redness relief eye-drop applicator in press fit squeeze bottle form according to the present invention;

FIG. 2 is bottom perspective view of the device of FIG. 1

FIG. 3 is a side perspective view of the device of FIG. 1

FIG. 4 1 is a perspective view of the combined cigarette lighter and redness relief eye-drop applicator in a thumb slide form according to the present invention;

FIG. 5 is a perspective view of the eye-drop applicator slid out separate from the device of FIG. 4

FIG. 6 is a side perspective of the device of FIG. 4

FIG. 7 is a bottom perspective of the device of FIG. 4 showing the hollow housing slide for FIG. 5

FIG. 8 is a perspective view of the combined cigarette lighter and redness relief eye-drop applicator in another press fit squeeze bottle form according to the present invention;

FIG. 9 is a side perspective of the device of FIG. 8

FIG. 10 is a perspective view of the combined cigarette lighter and redness relief eye-drop applicator in a non-removable pop-tab form according to the present invention;

FIG. 11 is a side perspective of the device of FIG. 10

FIG. 12 is a bottom perspective of the device of FIG. 10

FIG. 13 is a perspective view of the combined cigarette lighter and redness relief eye-drop applicator in a non-removable squeeze bottle form according to the present invention;

FIG. 14 is a side perspective of the device of FIG. 13

FIG. 15 is a bottom perspective of the device of FIG. 13

FIG. 16 is a perspective view of eye-drop applicator of the device of FIG. 4

### DESCRIPTION OF PREFERRED EMBODIMENTS

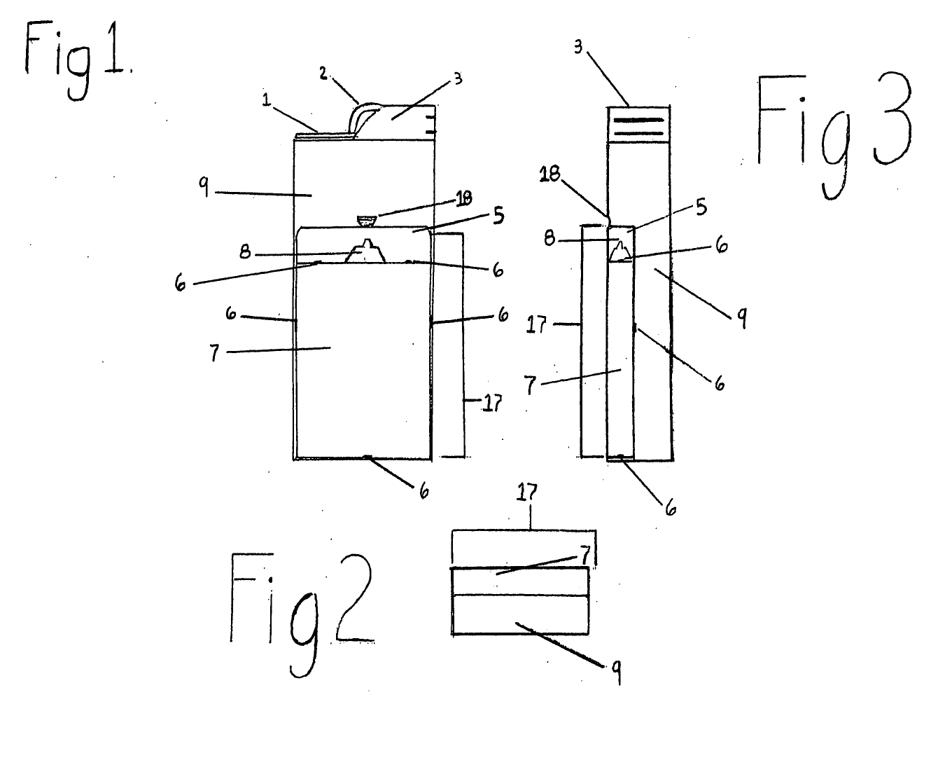

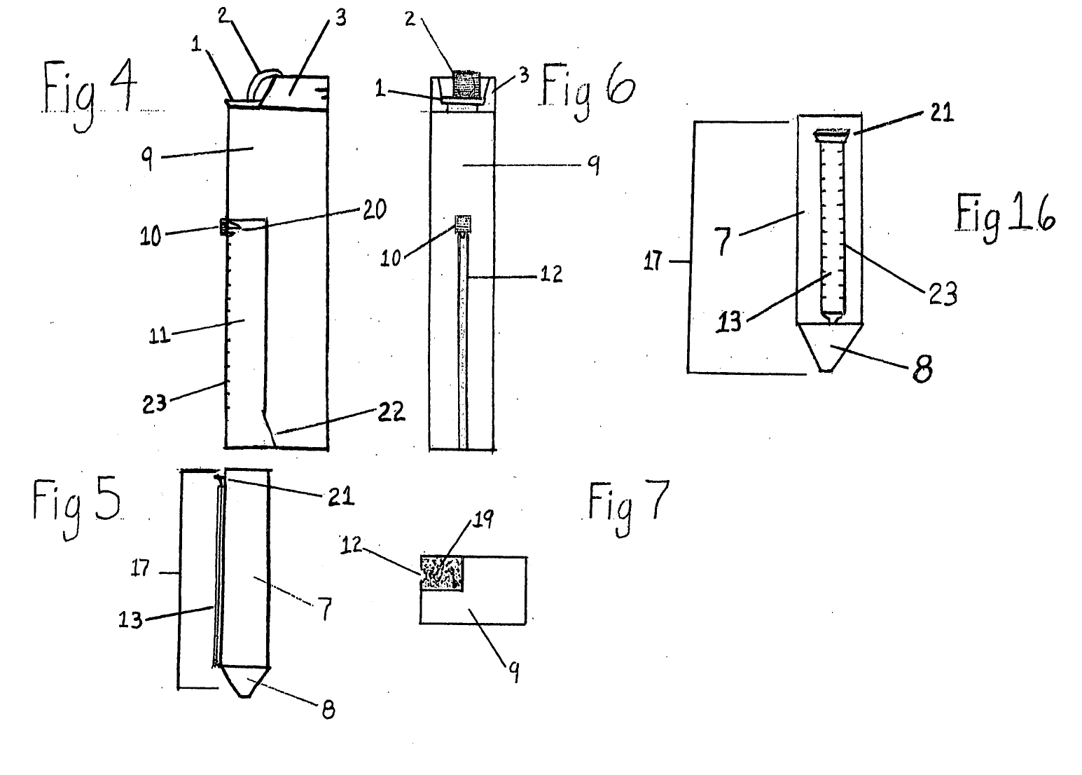

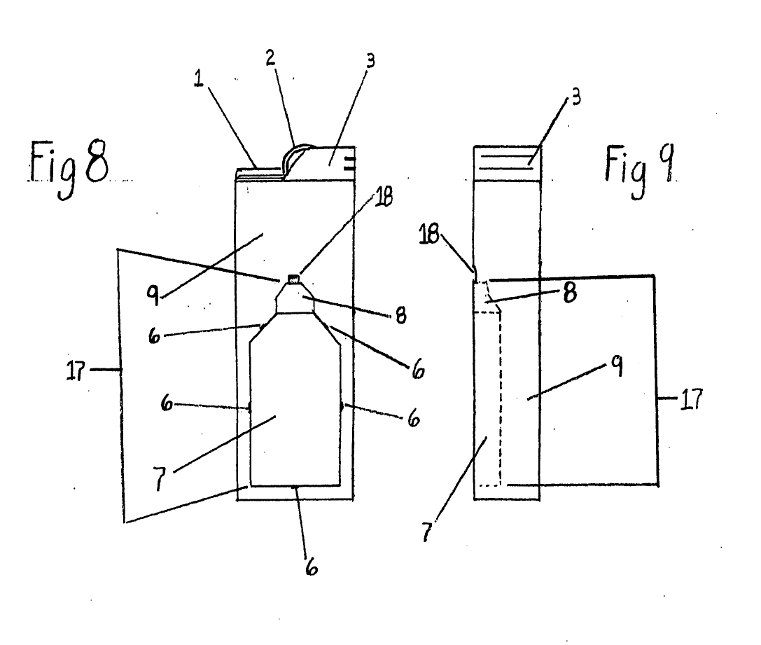

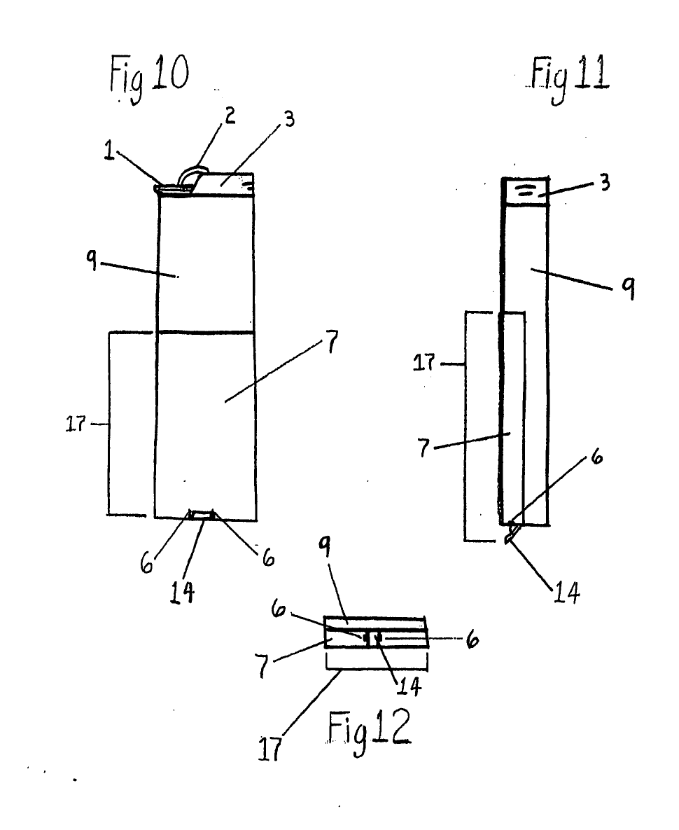

Referring now to the drawings, where like numerals represent like parts throughout, FIGS. 1 through 16 illustrate a combined cigarette lighter and eye-drop applicator device. The cigarette lighter 9 housing unseen fuel reservoir includes a lighting mechanism 1 and a flint wheel 2 used for igniting the flame, an electronic start lighting mechanism could also be utilized for igniting the flame and flame housing 3 containing the ignition apparatus (not shown). The fuel reservoir is to be disposable (not shown). The eye-drop applicator 17 includes a solution reservoir 7 (to be disposable) adapted to hold a predetermined supply of redness-reliever eye solution and a dispensing mechanism 8, 14 for dispensing the eye solution from the solution reservoir 7 set in cigarette lighter housing 9 alongside the fuel reservoir(not shown). The dispensing mechanism 8 and 14 includes a discharge orifice (not shown) at tip of dispensing mechanisms 8 and 14 to discharge the eye-drops. The dispensing mechanisms 8 and 14 may be a simple squeeze bottle applicator tip 8 or a pop tab applicator tip 14. The fluid reservoir 7 may be press-fit secured by plastic press-clips 6 in fuel housing of the lighter 9, thumb slide mechanism 10 (seen in FIGS. 4,5,6,7 and 16) secured in hollow housing of lighter 9, or firmly secured in housing 9 for non-removable use (seen in FIGS. 10,11,12,13,14 and 15).

Referring specifically to FIGS. 1, 2, 3, 8 and 9, the thumb indent 18 is located atop the eye-drop applicator 17 for easy removal from cigarette lighter housing 9 and release from plastic press-clips 6. Shown in FIGS. 1 and 3 there is a plastic cover cap 5 anchored to top of fluid reservoir 7 by plastic press-clips 6, forming the eye-drop applicator 17. The plastic cover cap 5 is to prevent unwanted discharge of fluid. FIGS. 8 and 9 function very similarly to FIGS. 1, 2 and 3. In FIGS. 8 and 9 eye-drop applicator 17 is further inset into cigarette lighter housing 9 secured by plastic press- clips 6 and squeeze bottle applicator tip 8 fits directly into thumb indent 18. Due to inset of eye-drop applicator 17 into cigarette lighter housing 9 there should be no unwanted discharge of fluid.

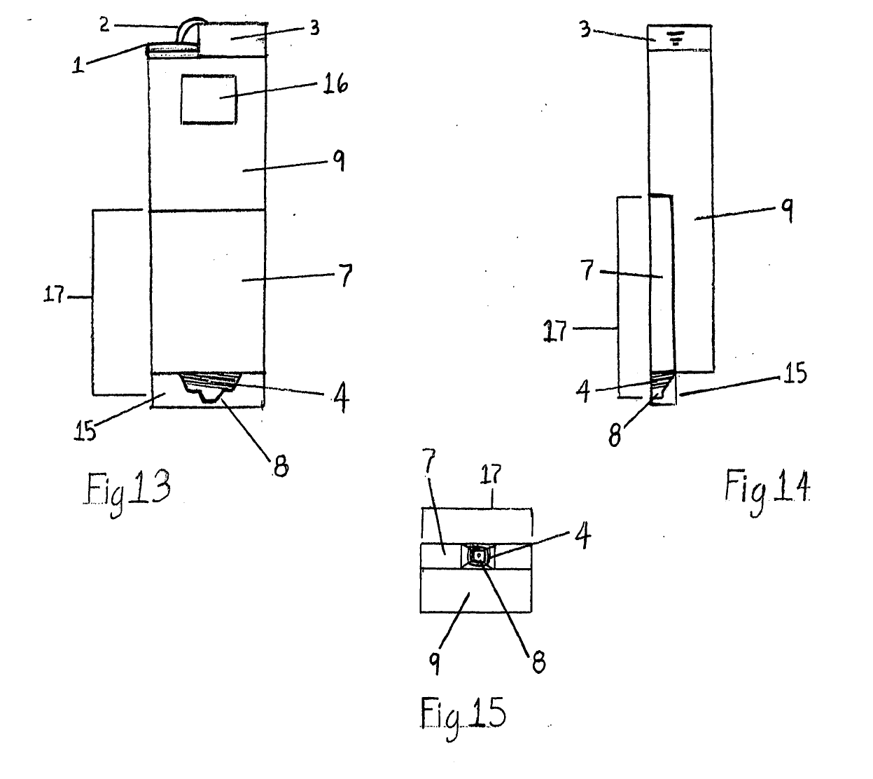

FIGS. 10, 11, 12, 13, 14 and 15 depict a non-removable eye-drop applicator 17. FIGS. 10, 11 and 12 have a pop-tab applicator tip 14 located on bottom of fluid reservoir 7, secured by press-fit clips 6 located on either side of pop-tab applicator 14. FIGS. 13, 14 and 15 have squeeze bottle applicator tip 8 located on bottom of fluid reservoir 7. Located on base of squeeze bottle applicator tip there are threads 4 where a threaded plastic cover cap 15 screws onto. Located on external portion of cigarette lighter housing 9, above the fluid reservoir 7 there is and identification plate 16, used for personalization of embodiment or for placing the owner's initials proving ownership of the embodiment, which should be noted as a very common problem among smokers.

FIGS. 4, 5, 6, 7 and 16 depict a thumb slide apparatus 10 which slides down the thumb slide track 12, used to remove the eye-drop applicator 17 from the eye-drop applicator housing 19. The eye-drop applicator 17 is held in place by friction bars 23 located on inside of eye-drop applicator housing 19 and on top of slide bar 13. The friction bars 23 keep eye-drop applicator 17 from falling out of eye-drop applicator housing 19. On the backside of thumb slide apparatus 10 there is a female connector 20 which connects to a male connector 21 located on top of the slide bar 13. Thumb slide apparatus 10 is then used to slide out eye-drop applicator 17 from the eye-drop applicator housing 19 and also used to retract eye-drop applicator 17 back to stationary carrying position. Release opening 22 located on bottom of eye-drop applicator housing 19 is then utilized to completely remove eye-drop applicator 17 in order for the user to freely administer the redness relief eye-drops, once thumb slide apparatus 10 has been slid all the way down to the release opening 22, once eye-drops have been administered user then re-attaches male connector 21 back into female connector 20 and then uses thumb slide apparatus 10 to slide eye-drop applicator 17 back to stationary carrying position.

We claim:

1. An apparatus comprising:
(A) A cigarette lighter device having a housing member and a lighting mechanism, said housing member having a bottom cavity portion; and
(B) An eye-drop applicator device for discharging eye-drops from an eye-drop reservoir, said eye-. drop applicator having a discharge device for controlling amount of eye-drops from the device, said device secured to the housing cavity

2. The apparatus of claim 1 wherein the eye-drop applicator is adapted to be removed and then secured to lighter by plastic press-fit clips in cavity portion.

3. The apparatus of claim 1 is secondly adapted to be a non-removable eye-drop applicator from the cavity portion.

4. The apparatus of claim 1 is thirdly adapted to be removed by thumb slide and secured by friction bars in the cavity portion.

5. The apparatus of claim 1, claim 2 and claim 3 further having a visible orientation so the entirety of the eye-drop applicator and the discharge mechanism is revealed to the user.

6. A combination cigarette lighter and eye-drop applicator referring to claim 4 which comprises:
(A) A slide-out mechanism comprising of a thumb slide allowing the eye-drops to be removed and then secured in housing by friction bars.
(B) Friction bars placed in cavity portion with female end to secure eye-drops in hollow housing. The male end is located on end of eye-drop applicator which in turn secures device in cavity portion. Eye-drops are to be removed and disposed of when fished, giving the user the option to use the lighter and eye-drops together or separate.

7. Eye-drops comprising of redness reliever eye solution used in:
(A) A relief of minor eye irritations caused by smoke or allergies, soothes and lubricates eyes relieving redness and itching.
